(19) United States
(12) Patent Application Publication
(10) Pub. No.: US 2016/0296799 A1
(43) Pub. Date: Oct. 13, 2016

(54) Wearable detector for the detection of parameters related to a physical activity

(71) Applicants: Davide Macagnano, Oulu (FI); Andrea Fabrizio Rinaldo, Gessate-Milano (IT)

(72) Inventors: Davide Macagnano, Oulu (FI); Andrea Fabrizio Rinaldo, Gessate-Milano (IT)

(73) Assignee: XMETRICS SPORTS LTD, Gerrards Cross, Buckinghamshire (GB)

(21) Appl. No.: 15/103,865

(22) PCT Filed: Dec. 17, 2014
(86) PCT No.: PCT/IB2014/002858

§ 371 Date: Jun. 11, 2016

(30) Foreign Application Priority Data
Dec. 20, 2013 (IT) MI2013A002171

(51) Int. Cl. A63B 24/00; A61B 7/00; A61B 5/145; A61B 5/01; A61B 5/00; A61B 5/0205

(52) U.S. Cl. CPC A63B 24/0062; A61B 5/6803; A61B 5/0205; A61B 5/14542; A61B 5/0022; A61B 5/01; A61B 7/00; A61B 5/486; A61B 5/02438

(57) ABSTRACT

A wearable detector for the detection of parameters related to a physical activity includes a support wearable on the user's head, a sound signal reproducing system connected to the support, an accelerometer, a gyroscope, a wireless signal receiving-transmitting device, and a control unit operatively connected to the sound signal reproducing system, to the accelerometer, to the gyroscope and to the receiving-transmitting device at least in order to manage data streams between those devices, wherein the support has a sealed chamber where at least the accelerometer and/or the gyroscope are housed, the support having such an arrangement that, in the worn condition, the chamber is placed substantially at the back of the user's head.

### TECHNICAL FIELD

The present invention relates to the field of detectors wearable by users for the detection of parameters related to a physical activity of the user.

### STATE OF THE ART

Detectors wearable by users for the detection of parameters related to a physical activity of the user are known in the state of the art.

Depending on the circumstances such detectors comprise sensors that are integrated in different ways in the device allowing parameters of interest to be detected.

A particularly interesting field of use for such detectors is the biomedical one, where they may be used for monitoring some parameters of a patient/user.

Another particularly interesting field which the present invention mainly relates to is the sports field, particularly the swimming field: during the several training phases it may be particularly useful to monitor the training parameters, such to have information about the health condition of the athlete, about his/her training condition or about other parameters directly or indirectly related thereto.

Several types of detectors are known in the prior art.

A first known detector is the one described in JP2011212167 allowing biomedical parameters to be detected by a sensor fitted into the user's ear; particularly the detected parameters are blood pressure, body temperature and the number of heart pulses.

Although such device is useful, it is not fit for being used in the sports field, but on the contrary it is suitable for being used in the biomedical field.

The detected parameters are not related to the situation the user is facing (he/she may be at rest) and moreover they are too much limited for allowing it to be used in the sports field, where the physical activity (intensity, phases of the activity and so on) causes such parameters to change, even considerably.

Finally it has to be considered that the use in the sports field leads to stresses that are not compatible with those a device intended to operate in the biomedical field is arranged for.

Another known detector is the one described in EP2229880: in short it relates to a band for the user's head, that allows heart pulses, blood oxygenation and acceleration of the patient to be detected.

In this case the device could be also suitable for being used in sports, however the detectable parameters are reduced in number and they do not allow all the parameters related to the physical activity to be monitored accurately and completely.

Moreover the sensors are mounted on the band and they are subjected to disturbances that cause the detection to be less accurate: for example among such disturbances it is possible to identify:
a displacement of the band from its position (the band cannot be too much tight on the head and in case of jerks—as in the case of running or swimming—it may easily move from its ideal position) and/or
the presence of a layer of sweat between the sensors and the user's skin
the presence of environmental noises/disturbances (e.g. rain, wind, sudden changes of temperature and the like).

Still another example is described in US 2008/0154098 describing an oximeter for detecting oxygenation and pulses, complete with an accelerometer and a thermometer integrated into a single device and having the possibility of a wireless interface with another mobile device.

However such device has some limits as regards its poor wearability; moreover the detectable data do not allow parameters of interest for the purposes of the invention to be detected.

Another known device is described in US 2009/0097689: mainly it is an audio peripheral unit with the possibility of attaching external sensors. Although it may be used in sporting activities, the sensors provided thereon can lead to disturbances and distorted or less accurate measurements, since they are easily subjected to external disturbances.

The same thing practically is valid also for the device described in US 2012/0274469 and for those described in WO 2010/054863 and US 2009/0105548.

### OBJECTS AND SUMMARY OF THE INVENTION

The aim of the present invention is to overcome the prior art drawbacks.

Particularly it is the aim of the present invention to provide a detector wearable by users for the detection of parameters related to a physical activity of the user capable of providing accurate measurements, the possibility of monitoring a plurality of signals and parameters and capable of interacting with a trainer or a coach and of listening to music or the like if necessary.

By means of the device of the invention it is possible to detect parameters both of the biometric type and of the biomechanical type during athlete training, with a particular reference to disciplines such as swimming, running, cycling, cross-country skiing.

While the sensed information can improve the sport experience by enabling the detection of data currently not available on other prior art devices, e.g. efficiency of the breathing phases, it also allows for the identification of dangerous situations arising during the activity, e.g. risk of drowning/collision, and the notification to user and/or other subjects monitoring the ongoing of the activity.

By means of the device of the invention it is possible to detect both the heart rate and the respiratory rate without sports activity-related disturbances that could make the measurements inaccurate.

The idea at the base of the invention is to provide a wearable detector for the detection of parameters related to a physical activity comprising of at least one or some of the following:
a support wearable on the user's head,
sound signal reproducing means connected to the support
one or more microphones
an accelerometer
a gyroscope
a magnetometer
one or more LED lights
one or more temperature sensors
a wireless signal receiving-transmitting device
a control unit operatively connected at least to the sound signal reproducing means, to the accelerometer, to the gyroscope, to the magnetometer, the LED lights and to the receiving-transmitting device at least in order to manage data streams between said devices.

For simplicity the term “control unit” as used herein may include one or more amplifiers, filters, comparators, microcontroller and circuits used to operate the sensors and compute operations.

According to the invention the support comprises a sealed chamber wherein at least the accelerometer and the gyroscope are housed, said support having such an arrangement that, in the worn condition, said chamber is placed substantially at the back of the user's head.

After several studies the Applicant has noted that the fact of placing even only the two sensors above mentioned (accelerometer and gyroscope) on the head, at a sealed chamber placed at the back of the user's head, allows the swim movements to be detected more accurately (particularly turn and swimming time).

Such data are also easy to be used as a source for obtaining other “derived” data such as the swim efficiency, the turn efficiency, the exerted force, by placing the accelerometers-gyroscopes in different locations (wrist, leg, trunk).

The Applicant has noted that due to the nature of the swimming movement it could not be possible to detect all the main parameters (it is impossible to detect the reverse of direction, the turn moment, the number of breaths and therefore to derive data about the turn efficiency) or it could not be possible to do it with the same accuracy (some movements would generate smaller accelerations).

Besides the swimming, just mentioned, the Applicant has further understood that also for other sports the positioning of the sensors is strategic: the movement of the head follows the movement of the trunk and it is less subjected to big displacements.

The encumbrance of the detector of the present invention further allows the athlete not to wear other devices than that provided for monitoring the necessary parameters.

According to a further advantageous characteristic, at least the antenna of the wireless signal receiving-transmitting device is placed in the sealed chamber at the back of the head, preferably at the occipital bone.

With reference again to swimming, the fact of positioning antennas for transmitting/receiving data in wireless mode is optimal when it is placed at the back, since, except for the “backstroke”, it would be always out of water, therefore allowing the “Bluetooth” standard (or similar) to be used without interferences (remember that such standard is not compatible with the underwater use just because of its operating frequency).

On the contrary as regards other standards the signal would be attenuated less therefore requiring less necessary power.

According to a further particularly advantageous characteristic the detector of the invention comprises at least one auricular device connected to the support and intended to be inserted in the ear cavity of a user: the auricular device houses at least one microphone for detecting a sound signal in the ear cavity of the user.

As a combination the sound signal reproducing means is a bone transducer for the bone conduction of sound signals, connected to said support and intended to be placed in contact with the bones of the skull of a user, outside of his/her ear cavity and therefore housed outside of the auricular device, that therefore is preferably free from any sound signal reproducing means.

The idea of moving the sound transducer for transmitting the sounds to the user outside of the ear cavity, by using the bone conduction and on the contrary the idea of fitting a microphone inside the auricular device for detecting sounds into the body of the user is particularly advantageous since it allows sounds indicative of the breathing or the voice of the user to be detected without being affected by sports activities, by weather conditions or the like.

At the same time the fact of using the bone transducer for the bone conduction of the sounds is particularly advantageous since this type of sound signal transmission (with respect to the one that may be obtained by the loudspeakers usually integrated in the earphones), is less subjected to environmental disturbances, above all in the case of swimming.

Another object of the present invention is a method for retrieving and/or monitoring physical parameter of a user, for example breathing rate or heart rate.

Further advantageous characteristics are the object of the annexed claims, that are an integral part of the present description.

### BRIEF DESCRIPTION OF THE DRAWINGS

The invention will be described below with reference to non-limiting examples, provided by way of example and not as a limitation in the annexed drawings. These drawings show different aspects and embodiments of the present invention and, where appropriate, reference numerals showing like structures, components, materials and/or elements in different figures are denoted by like reference numerals.

FIG. 1 is a perspective view of the device of the invention;

FIG. 2 is a perspective view of a detail of the device of the invention;

FIGS. 3 and 4 are perspective views of the device of the invention in the worn condition;

FIG. 5 is a perspective view of an embodiment of the device of the invention;

FIG. 6 is an enlarged detail of FIG. 5;

FIG. 7 is perspective view of another embodiment of the device of the invention.

### DETAILED DESCRIPTION OF THE INVENTION

While the invention is susceptible of various modifications and alternative forms, some disclosed relevant embodiments are shown in the drawings and will be described below in detail.

It should be understood, however, that there is no intention to limit the invention to the specific embodiment disclosed, but, on the contrary, the intention of the invention is to cover all modifications, alternative constructions and equivalents falling within the scope of the invention as defined in the claims.

The use of “for example”, “etc”, “or” indicates non-exclusive alternatives without limitation unless otherwise defined.

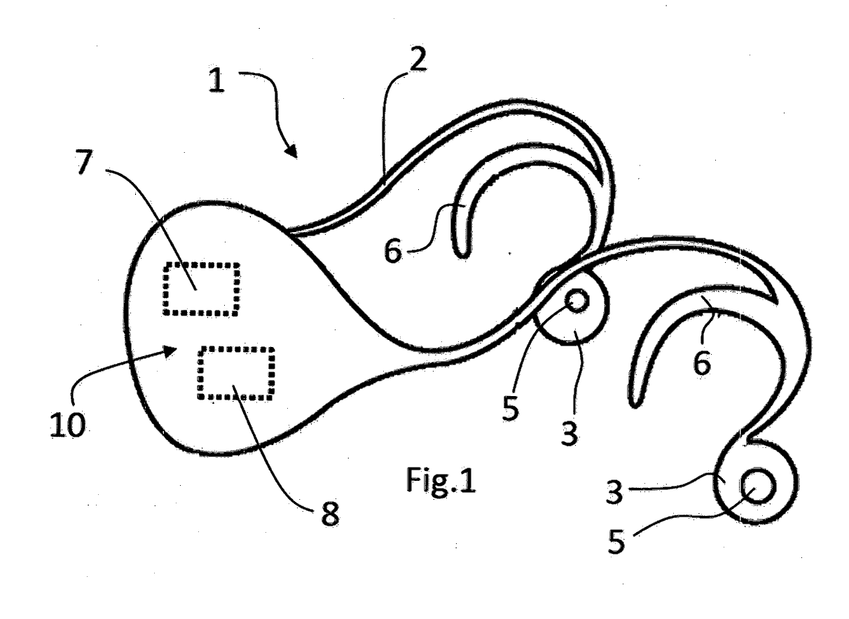

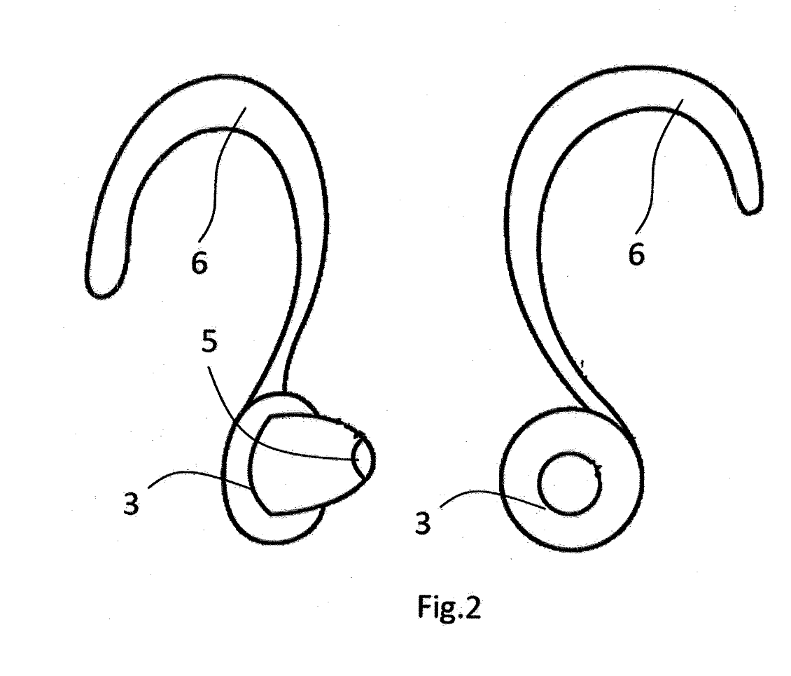

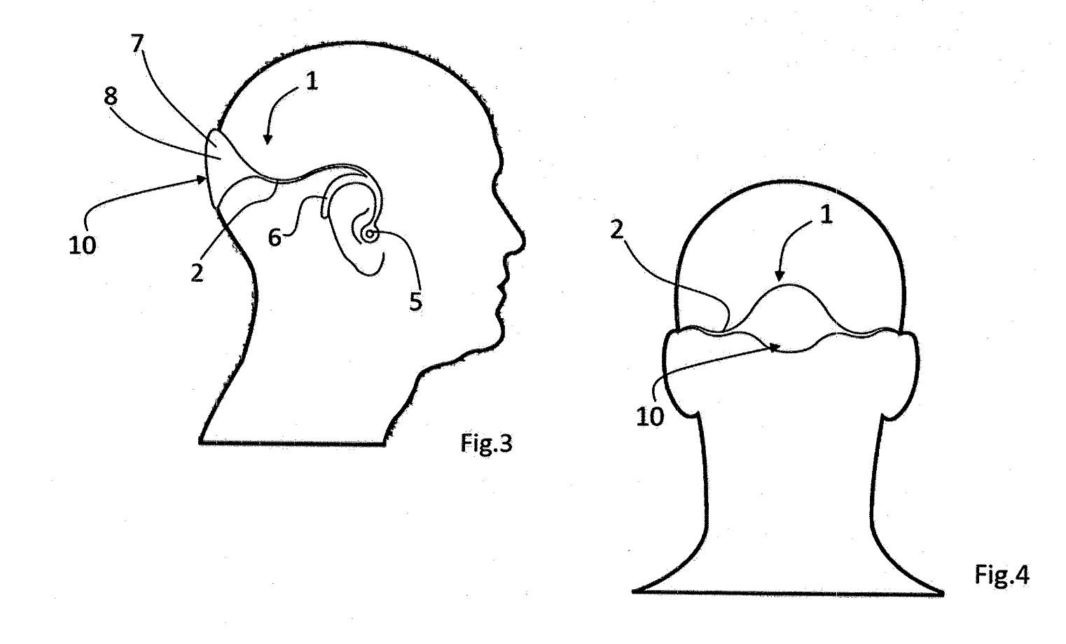

The use of “including” means “including, but not limited to,” unless otherwise defined. With reference to the annexed FIGS. 1-4 and 5,6 they show a first embodiment of a wearable detector 1 for the detection of parameters related to a physical activity comprising a support 2, preferably an arch support, wearable on the head of a user, two auricular devices 3 connected to the arch support 2 and intended to be inserted in the ear cavity of a user.

Support 2 in some embodiment is a flexible support, like a wire or similar.

It must be noted since now that with the term “auricular device” it is, here and in the claims, intended an earplug that is suitable for being housed, in use condition, inside the auricular cavity of a user, despite of its functional components

Now it has also to be noted that the embodiment shown in the drawings 1-4 is an advanced first embodiment.

The basic embodiment of the detector of the invention comprises
one support 2 wearable on the user's head, for example thanks to suitable supporting or anchoring means
sound signal reproducing means 6 connected to the support 2
an accelerometer
a gyroscope
a wireless signal receiving-transmitting device 8
optionally one or more LED lights 90
optionally a magnetometer
a control unit 7 operatively connected to the sound signal reproducing means 6, to the accelerometer, to the gyroscope, to the LED lights 90 and to the receiving-transmitting device 8 at least in order to manage data streams between said devices.

The support 2 comprises a sealed chamber 10 wherein at least the accelerometer and the gyroscope and preferably part of the LED lights 90 as well as the wireless signal receiving-transmitting device 8 are housed.

The sealed chamber 10 is placed on the support 2 such that, when in use, it is placed substantially at the back of the user's head.

This allows the advantages set forth above to be achieved and no further reference is made thereto.

Preferably, such as in the shown example, in the worn condition, the sealed chamber 10 is placed substantially at the occipital bone of a user's head.

This leads to a further advantage, related to the fact that, when swimming, such region is the region projecting most from the water (in the emersed condition) for most of the time of the swim.

With reference to such chamber 10 it has to be noted that it may also be formed by embedding the functional components (accelerometer, gyroscope, magnetometer and possibly the LED lights, the wireless signal receiving-transmitting device 8 and the battery or so on) completely or partially into the material of the support 2.

Thus an arrangement of the detector may be provided wherein there is provided more than one chamber 10 housing at least the gyroscope and/or the accelerometer and/or magnetometer or a situation wherein a sealed chamber 10 is divided into many sub-chambers divided by walls but in communication with each other (even only through the areas for the passage of the cables).

For example this may be obtained when said material of the support 2 is rubber or a plastic polymer, by pouring the material above the functional components, which therefore are embedded in the support 2 into corresponding sub-chambers that therefore have to be intended as sealed ones, even if they are in communication with each other by the cables that transport the operating signals among the several functional components.

In the shown advanced first embodiment of FIG. 1-4, as it will be described in details below, the sound signal reproducing means 6 is a bone transducer, but more generally it may be a loudspeaker, such as those in the usual earphones for listening to the music. In this last case the reproducing means in the form of a loudspeaker will be placed on one of the auricular devices 3, such that it can be inserted in the ear cavity of a user. On the contrary, the additional advantages related to the use of the bone transducer 6 will be more clear soon.

In the shown advanced embodiment, at least one of the auricular or earplug devices 3 houses a microphone 5 for detecting a sound signal in the ear cavity of the user.

The microphone 5 therefore is placed in the portion of the auricular or earplug device 3 intended to be inserted in the ear cavity of the user.

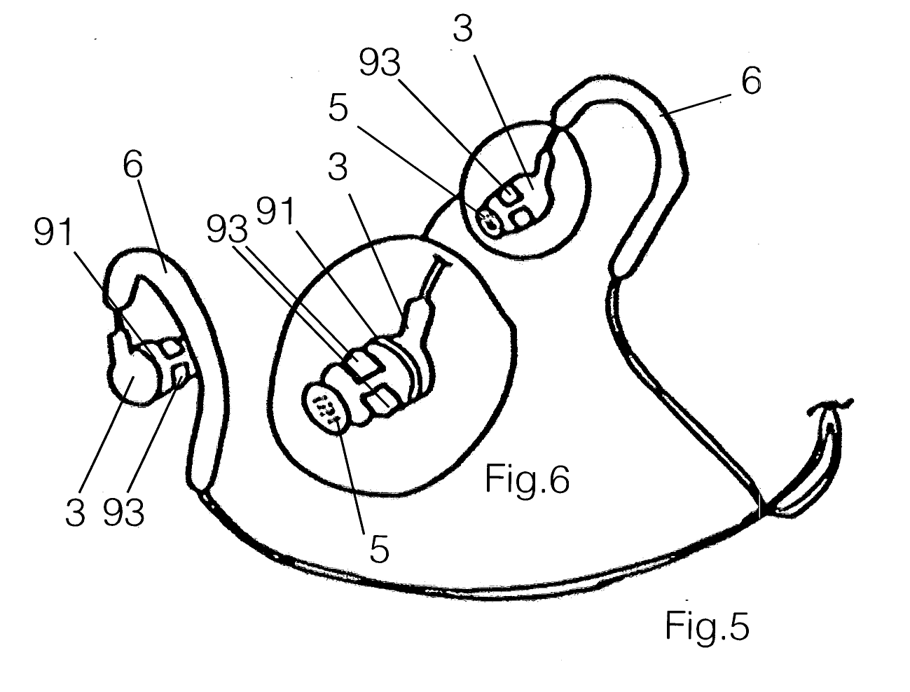

To this extent reference can be made to FIG. 5 and the enlarged particular of FIG. 6.

The earplug 3 houses the microphone 5 with its active portion (the sensing portion of the microphone through with it retrieves the sounds) facing the internal part of the auricular cavity of the user.

To this extent the microphone 5 is housed at the free end of the auricular device 3, with the active portion placed at the opposite than the earplug sealing 91.

The latter can be in the form of a ring or a crown, that protrudes circumferentially around the earplug 3 in order to establish a sealing against the walls of the auricular cavity of a user, when in place; to this extent the ring can be in rubber or similar flexible material: in this way the internal auricular cavity of a user became isolated from the outside environment, preferably both in terms of sealing against liquids and acoustic isolation.

This allows two main advantages: first, the microphone 5 is placed in the dry environment of the auricular cavity of the user, even if on the outside the environment is wet (such for example when the device is used in swimming activities); second, the microphone can retrieve sounds in such a cavity without being disturbed by external environment sounds.

Furthermore, in an embodiment, also further biological sensors 93 are provided on the earplug 3, particularly in the portion of the latter that is comprised between the free end (having the microphone active portion) and the sealing 91; in this way, when in use, the biological sensors 93 senses the inside of the auricular cavity of the user, retrieving biological parameters without being influenced by the outside environment, similar to what has been discussed for the microphone 5.

Different kinds of biological sensors 93 can be used in practice: for example thermometers or similar temperature sensing means, oximeters and others.

For what concerns the microphone 5, its aim is to detect more accurately the respiratory information, e.g. rate, volume of oxygen/CO2 exchanged per breathing cycle and so forth, without affecting or jamming the detection by external noises.

Indeed, to improve performance during the practice of sports activities one key factor to be studied is breathing.

Amongst the different metrics that can be associated with it, rate and the amount of oxygen and CO2 exchanged during the breathing cycle are essential information to improve the athletes' potentials.

This is particularly true when this information is correlated with other physiological information, e.g. heart rate, tidal volume, gender, height, and so forth.

According to this, in one embodiment the method of the invention relies on the recorded acoustic signal of breath by the microphone placed in the ear canal.

This is used to measure the breathing rate as well as the depth and duration of the phases during the breathing cycle, their associated energy, similarity/dissimilarity to recorded patterns and so forth.

To this end, after filtering out the ambient noise from the recoded signal, a voice activity detection (VAD) technique, or similar, is used to segment the signal in breathing cycles and the corresponding phases, i.e. inhale/exhale phase.

Then the energy of the signal associated to each phase of the breathing cycle is compared versus the physiological limit of the users computed on bases of their tidal volume.

Notice that this could be either obtained from practicing deep breath cycles before the activity from which the lung capacity could be inferred, or as an input parameter computed on the basis of tests with spirometers.

One possible result of the comparison aims at providing the user with a feedback (e.g. audio) on the quality of the entire breathing or only some key aspect of it, e.g. exhalation.

Also this information can be use to notify the user whether he should increase/decrease the rate, intensity of the activity.

Clearly the same kind of analysis can be performed a posteriori on external devices on the basis of the data recorded by the devices.

Still by means of the microphone 5 it is provided to obtain the voice of the athlete such that, once processed/filtered, it can be transmitted to the coach.

Moreover, using the microphone installed in the earplug jointly with a Voice Activity Detection (VAD) algorithm implemented on the processing unit, it is possible to detect commands from the users.

These commands can be used to trigger actions, e.g. alarm signals and/or provide feedbacks from the user to external entities, e.g. coach or lifeguards, either by means of the LED lights and/or through the wireless communication module.

For instance, in presence of potentially dangerous situations, e.g. a sudden stroke by the user, the device can detect/interpret designated voice commands, e.g. “help”, and activate the visual (LED) and or radio signaling associated with it.

More in general voice commands can be used to activate light indicators embedded onto the device or connected to it by wired/wireless link.

As an example, this could be used in cycling to notify the direction in which the user intends to turn.

The detector 1 in the advanced embodiment comprises at least one bone transducer 6 for the bone conduction of sound signals, preferably two as in the attached figures.

Such bone transducers 6 are known in se and therefore it is not necessary to dwell on their operation.

By means of such bone transducers 6 the athlete will be able, when training, to listen to the music coming from an external source (e.g. smarthpne/mp3 reader/smartwatch) or from the detector 1 itself, as well as he/she will be able to communicate, by means of the microphone in the earplug, and to receive feedback in real-time from the coach through radio/wireless communication or from the device itself that is capable of processing in real-time the data in order to give a first instruction about the training and the health condition of the athlete.

Obviously it has to be noted now that instead of the two auricular or earplug devices and of the two bone transducers it may be possible to provide only one of the former and/or of the latter.

As regards the control unit 7 and the transreceiver unit 8 they are operatively connected with each other: in the advanced embodiment the control unit 7 is further operatively connected to the microphone 5 and to the bone transducer 6 such to receive/transmit the signals and to process them, if any.

As an optional feature, recording means, such for example a RAM can be provided, in order to record data retrieved by the microphone and/or other components of the device 1 during operation.

The two units 7 and 8 in some embodiments can be integrated together, without this changing their functionality.

As regards the transreceiver unit 8 it is intended for transmitting/receiving wireless signals, such as for example radio, bluetooth signals or the like.

In the preferred embodiment the units 7 and 8 are housed together with a power supply electric battery in the sealed chamber 10 of the arch, intended, in use, to be placed approximately at the back of the user's head.

In advanced embodiments the detector 1 comprises also other types of sensors, for example one or more sensors of the biomechanical type such as three-axes accelerometers, gyroscopes and magnetometers, and GPS.

These biomechanical sensors may be placed inside the auricular device 3 or outside it, for example on the arch 2 or in the chamber 10.

The biomechanical sensors serve for detecting the attitude of the device, movements, efficiency, velocities, forces generated and the displacements of the athletes as well as parameters such as number and the frequency of the strides when running, or the number of strokes and lengths when swimming or the frequency and the length of the strides in cross-country skiing, braking or turning while cycling and so forth.

The fact of placing the biomechanical sensors on the detector 1, worn on the athlete's head, allows parameters to be detected more accurately such as the number of breaths when swimming or the moment of turning, further reducing the effect of other secondary movements that can create disturbances when training and making the instrument less subjected to disturbances deriving from the surrounding environment (e.g. temperature, rain, noises and the like) and from the physical activity itself (e.g. perspiration or the like).

Also the placement of the device on the head of the user allows the automatic detection of potentially dangerous situations/events.

For instance, in swimming the attitude information recovered from the sensed data can be used to recognize whether the user is drowning, e.g. by detecting if the face is submerged for a prolonged time or whether the user is sinking more than a predefined threshold.

The detection of such events can then be used to trigger visual (LED), radio or even audio signals.

As already said, as an alternative or in combination, the detector 1 comprises also other types of sensors, for example one or more sensors of the biological, preferably biomedical, type 93, such as a “pulse oximeter” (for detecting blood oxygenation, heart rate, blood pressure and the like) one or more thermometers, an oximeter, a blood-oxygen monitor or the like.

In one preferred embodiment, two temperature sensors, such thermometers or the like, are provided, one internal to the ear canal (in the earplug or auricular device 3) and another one, external to it.

In some embodiment, some biomedical sensors are not in the auricular device 3, but near to the latter, so as to face the external environment when in use.

In order to improve the detection of essential parameters in some applications it is provided that the evaluations coming from the oximeter can be combined with those coming from the microphone (e.g. for detecting heartbeats) or data coming from accelerometers for filtering a part of the noise due to the movement.

In combination or as an alternative to unit 8 it is provided to install a storage unit wherein the data detected by the sensors are to be stored.

Even in this case the method of the invention provides for using the biomedical information to trigger visual/radio signaling of different nature.

For instance in a training session with a team of athletes the LED lights (and or the radio) could be used to indicate on real time in a concise manner parameters of interest, e.g. hearth rate, velocity, progress of the training, efficiency of the breathing. The same applies for the other biomedical data sensed from the device.

Also alert signaling can be activated in case of danger, e.g. internal temperature dropping, pulse rate below/over safety regions and so forth.

This can be essential to detect and even prevent situation of danger commonly encountered when a large number of athletes needs to be monitored at the same time, e.g. swimming competitions in open waters.

As an alternative or in combination the above the device comprises also a speaker for audio feedback in case dangerous events have been detected by the device.

If the device is connected to an external device (smartphone or smartwatch or a remote computer for example) the latter can be provided with an additional software for processing the data.

In this case it is possible for the detector 1 to directly or indirectly use the additional sensors on the external device, such as for example the GPS, altimeter, thermometer, barometer.

In this case the wireless communication (for example) with an external unit can be directly given to the wireless module of the smartphone/smartwatch.

As an alternative a suitable module inside the device will be used as described above. The wireless communication module, together with the provision of a microphone/sound transmission by cranical bones will enable a bidirectional communication/feedback system between athlete and coach that will be able to handle more than one athlete at the same time.

In a different embodiment comprising some or all the features above the attitude information computed from the device is used to provide the user, during swimming activities, with remaining distance to complete their current lap.

This could be used to set up virtual races by the users or to provide blind users with warning messages in proximity of the end of the pool.

With reference to the latter use case, also proximity information from other swimmers can be inferred on the basis of the radio units included in the devices.

As an alternative or in combination to the embodiments it is considered that the radio signal emitted by the device can be utilized by an infrastructure/system to recover its location in the environment.

This information can then be provided on real time to the coach or the user itself. The latter case can be used, e.g. in presence blind users, to set up a system that helps to prevent collisions amongst swimmers and with obstacles, e.g. the sides of the pool.

In a different embodiment the device could comprise, in alternative or in combination to the realization described above, of a vibrating disk motor or similar.

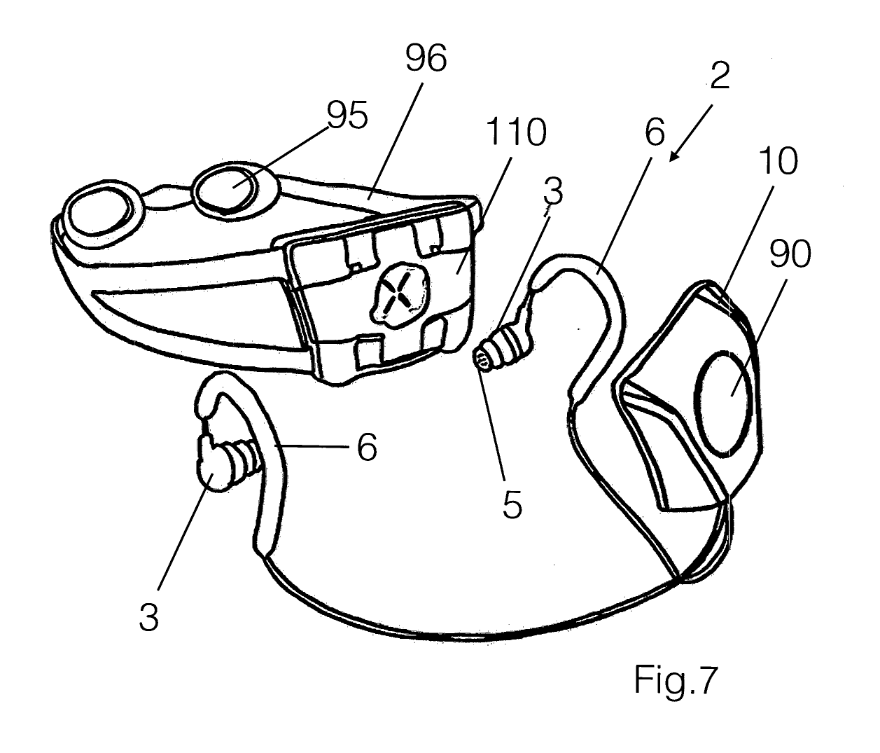

Another embodiment of the invention is shown in FIG. 7.

With the same numeral references are indicated the same parts of the previous embodiment.

In this embodiment the device comprises the support 2 which comprises on its turn also a pair of glasses 95, such for example swimming pool glasses.

The chamber 10 is removably coupled with a base 110 which is coupled to the glasses 95 by a retaining system, such for example a pair of elastic bands 96 or similar.

Another object of the present invention is a method for retrieving or detecting physical parameters of a user, such for example breathing rate, heart rate, blood oxygenation and similar.

In a first embodiment the method provide the step of using a detector as already described.

In a second independent embodiment, the method provide the step of acquiring a sound in the ear cavity of a user and analyzing such sound for retrieving the breathing rate and/or the efficiently of the respiration, i.e. the amount of oxygen inhaled and CO2 exhaled with respect to the tidal volume during the breathing cycle or the number of breathing acts that the user has performed in a certain time interval.

In a third embodiment the method provide the step of acquiring a sound in the ear cavity of a user and analyzing such sound for retrieving the heart rate or the number of heart beats that the user has performed, preferably in a certain time interval.

In a fourth embodiment the method combines the information on the breathing together with at least an head attitude information relating the user head attitude to provide the user with a feedback, e.g. audio, on the quality of the respiration activity. Then many variants to what described up to now are possible, all to be considered within the reach of the person skilled in the art in the light of the above description and therefore equivalent to what claimed below.

The invention claimed is:

1. A wearable detector (1) for detection of parameters related to a physical activity, comprising:
a plurality of devices comprising,
a support (2) wearable on a user's head,
a sound signal reproducing system (6) connected to said support,
an accelerometer (13),
a gyroscope (14), and
a wireless signal receiving-transmitting device (8); and
a control unit (7) operatively connected to the sound signal reproducing system (6), to the accelerometer, to the gyroscope and to the receiving-transmitting device (8) at least to manage data streams between said plurality of devices,
wherein said support (2) comprises a sealed chamber (10) where at least the accelerometer and/or the gyroscope are housed, and
wherein said support (2) has such an arrangement that, in a worn condition, said chamber (10) is placed substantially at the back of the user's head.

2. The wearable detector (1) according to claim 1, wherein said support has such an arrangement that, in the worn condition, said chamber (10) is placed substantially at an occipital bone of the user's head.

3. The wearable detector (1) according to claim 1, wherein said support (2) is an arch support wearable as a headphone on the user's head.

4. The wearable detector (1) according to claim 1, further comprising at least one auricular or earplug device (3) connected to said support (2) and configured to be inserted in an ear cavity of the user, wherein said auricular or earplug device (3) houses at least one microphone (5) for detecting a sound signal in the ear cavity of the user.

5. The wearable detector (1) according to claim 1, wherein the sound signal reproducing system (6) comprises a bone transducer (6) for bone conduction of sound signals, connected to said support and configured to be placed in contact with bones of the user's skull, outside of the user's ear cavity.

6. The wearable detector (1) according to claim 1, further comprising a georeferencing system.

7. The wearable detector (1) according to claim 4, further comprising at least one biomedical sensor.

8. The wearable detector (1) according to claim 7, wherein said at least one biomedical sensor is housed into the auricular device (3).

9. The wearable detector (1) according to claim 7, further comprising a storage unit for storing data detected by said at least one biomedical sensor.

10. A method of retrieving or detecting physical parameters of a user, such physical parameters comprising one or more of breathing rate, heart rate, or blood oxygenation, comprising:
providing a detector (1) having a plurality of devices comprising a support (2) wearable on a user's head, a sound signal reproducing system (6) connected to said support, an accelerometer (13), a gyroscope (14), and a wireless signal receiving-transmitting device (8), and further having a control unit (7) operatively connected to the sound signal reproducing system (6), to the accelerometer, to the gyroscope and to the receiving-transmitting device (8) at least to manage data streams between said plurality of devices,
wherein said support (2) comprises a sealed chamber (10) where at least the accelerometer and/or the gyroscope are housed, and
wherein said support (2) has such an arrangement that, in a worn condition, said chamber (10) is placed substantially at a back of the user's head; and
having the user wear the detector such that the chamber (10) is placed substantially at a back of the user's head.

11. The wearable detector (1) according to claim 6, wherein the georeferencing system is a GPS antenna.

12. The wearable detector (1) according to claim 7, wherein said at least one biomedical sensor is selected from the group consisting of a pulse oximeter, a thermometer, an oximeter, or a blood-oxygen monitor.
